(19) United States
(12) Patent Application Publication
(10) Pub. No.: US 2024/0050597 A1
(43) Pub. Date: Feb. 15, 2024

(54) RADIOLABELLED ALPHA-V BETA-3 AND/OR ALPHA-V BETA-5 INTEGRINS ANTAGONIST FOR USE AS THERAGNOSTIC AGENT

(71) Applicants: ADVANCED ACCELERATOR APPLICATIONS INTERNATIONAL SA, GENEVA (CH); ADVANCED ACCELERATOR APPLICATIONS (ITALY) SRL, POZZILLI (IT); NOVARTIS AG, BASEL (CH)

(72) Inventors: Valeria MUZIO, POZZILLI (IT); Antje WEGENER, GENEVE (CH); John Scott CAMERON, CAMBRIDGE, MA (US); Francesco DE CARLI, POZZILLI (IT); Paola BARDINI, POZZILLI (IT); Alessandro MAGRI, POZZILLI (IT); Mattia ROSSETTO, POZZILLI (IT); Daniela BARENGO, POZZILLI (IT)

(73) Assignees: ADVANCED ACCELERATOR APPLICATIONS INTERNATIONAL SA, GENEVA (CH); ADVANCED ACCELERATOR APPLICATIONS (ITALY) SRL, POZZILLI (IT); NOVARTIS AG, BASEL (CH)

(21) Appl. No.: 18/268,779

(22) PCT Filed: Dec. 20, 2021
(86) PCT No.: PCT/EP2021/086885

§ 371 Date: Jun. 21, 2023

(30) Foreign Application Priority Data
Dec. 21, 2020 (EP) 20216053.7

(51) Int. Cl. A61K 51/04

(52) U.S. Cl. CPC A61K 51/0455; A61K 51/0497; A61K 51/0402

(57) ABSTRACT

The present disclosure relates to αvβ3 and/or αvβ5 integrins antagonist radiopharmaceuticals and their use in a theragnostic approach for selection and therapy of human subjects with tumors overexpressing αvβ3 and/or αvβ5 integrins. In particular, the present disclosure relates to a pharmaceutical composition of αv ¹⁷⁷Lu radiolabeled αvβ3 and/or αvβ5 integrins antagonist, for use in treating tumors overexpressing αvβ3 and/or αvβ5 integrins in a human subject eligible for said treatment, wherein said subject has been selected for the treatment by PET/CT or PET/MRI or SPECT/CT or SPECT/MRI imaging with the same αvβ3 and/or αvβ5 integrins antagonist but with 68-Ga as radiometal for use as imaging agent.

### TECHNICAL FIELD

The present disclosure relates to αvβ3 and/or αvβ5 integrins antagonist radiopharmaceuticals and their use in a theragnostic approach for selection and therapy of human subjects with tumors overexpressing αvβ3 and/or αvβ5 integrins. In particular, the present disclosure relates to a pharmaceutical composition of a ¹⁷⁷Lu radiolabelled αvβ3 and/or αvβ5 integrins antagonist, for use in treating tumors or tumor vasculature overexpressing αvβ3 and/or αvβ5 integrins in a human subject eligible for said treatment, wherein said subject has been selected for the treatment by PET/CT or PET/MRI imaging with the same αvβ3 and/or αvβ5 integrins antagonist but with 68-Ga as radiometal for use as imaging agent.

### BACKGROUND ART

Integrins are heterodimeric receptors that are important for cell-cell and cell-extracellular matrix (ECM) interactions and are composed of one α and one β-subunit. These cell adhesion molecules act as transmembrane linkers between their extracellular ligands and the cytoskeleton and modulate various signalling pathways essential in the biological functions of most cells. Integrins play a crucial role in processes such as cell migration, angiogenesis, wound healing, haemostasis and oncogenic transformation. The fact that many integrins are also linked with pathological conditions has led them to become promising therapeutic targets. In particular, integrins αvβ3, αvβ5 are involved in angiogenesis and metastasis of solid tumors, making them potential candidates for cancer therapy, including those with a high unmet medical need, such as glioblastoma, head and neck cancer, colorectal cancer, breast cancer, small cell lung cancer, non-small cell lung cancer, malignant melanoma, pancreatic cancer, prostate cancer and brain metastasis.

Both αvβ3 and αvβ5 are expressed in various cell types such as endothelial cells, fibroblasts, epithelial cells, osteoblasts, and smooth muscle cells and are upregulated in endothelial cells undergoing angiogenesis. They are not only highly expressed on morphologically abnormal tumor vasculature, but also on tumor cells, including gliomas. Positron emission tomography (PET) using [18F]Galacto-RGD and validation by immunohistochemistry revealed αvβ3 expression in different solid tumors of patients but lack of expression in normal tissues (e.g. benign lymph nodes, muscles). In addition, it has been shown that activation of αvβ3 is required for metastasis in a breast cancer carcinoma model and that expression of αvβ3 and αvβ5 in tumor vasculature correlates with the malignancy of neuroblastoma. The selective upregulation of the αvβ3 and αvβ5 receptors in malignant glioma suggests a major role for this integrin in this type of cancer. Thus, due to their primary expression on activated endothelial cells, the αvβ3 and αvβ5 integrins are attractive targets for cancer therapy (Mas-Moruno et al, 2010).

The most prominent compound targeting integrins may be cilengitide, a cyclic RGD peptide that targets αvβ3 and αvβ5 integrins, developed for the treatment of glioblastomas and other tumors. Its features and potency boosted research in the last two decades towards a plethora of new small molecules able to antagonize integrins. However, despite great expectations, antagonists of αvβ3 and αvβ5 integrins that have entered clinical trials as antiangiogenic agents for cancer treatment, including cilengitide, have generally been unsuccessful. Nevertheless, integrins remain a potential treatment target for glioblastomas (Tolomelli et al, 2016).

Radiolabelled receptor-binding molecules are considered an important class of radiopharmaceuticals for tumour diagnosis and therapy. The use of radiolabelled molecules to target receptor-expressing tissues in vivo has stimulated a high level of interest in the field of nuclear medicine (Reubi, 2003). Peptides can be labelled for use with both Single Photon Emission Computed Tomography (SPECT) and Positron Emission Tomography (PET). Commonly used gamma emitters are 111In and 99mTc for use with SPECT imaging, while for PET imaging, peptides can be radiolabelled with positron emitting radionuclides, such as 68Ga, 18F and 64Cu. The 111In-labelled somatostatin analogue octreotide (OctreoScan™), the first approved radiopeptide for the imaging of several types of neuroendocrine tumors, has paved the way for the development of several other radiolabeled-peptides targeting different receptors.

Available literature data (Beer et al., 2007; Mena et al., 2014; Iagaru et al., 2014; Kim et al., 2012) in humans confirmed that 18F and 68Ga radiolabelled RGD peptides targeting αvβ3 and αvβ5 integrins are well tolerated.

Despite many therapeutic advances, several common tumors such as glioblastoma, head and neck cancer, colorectal cancer, breast cancer, small cell lung cancer, non-small cell lung cancer, malignant melanoma, pancreatic cancer, prostate cancer and brain metastasis, are still a frequent cause of death and new treatment approaches are needed.

In this context, it would thus be desirable to provide a novel theragnostic approach for selection and therapy of αvβ3 and/or αvβ5 overexpressing tumors.

The present disclosure relates to a theragnostic approach based on the use of a radiolabeled αvβ3 and/or αvβ5 integrins antagonist with (1) Gallium 68 (⁶⁸Ga) as a diagnostic to identify tumor lesions and select patients suitable for radioligand therapy (RLT) and (2) Lutetium-177 (¹⁷⁷Lu) for the treatment of these tumor lesions by (RLT), in particular on glioblastoma, head and neck cancer, gastroesophageal adenocarcinoma, colorectal cancer, breast cancer, small cell lung cancer, non-small cell lung cancer, malignant melanoma, pancreatic cancer, in particular pancreatic ductal adenocarcinoma, gastric cancer, prostate cancer or brain metastasis, in particular brain metastasis from breast cancer and melanoma.

### SUMMARY

The present disclosure relates to a pharmaceutical composition of a radiolabelled αvβ3 and/or αvβ5 integrins antagonist, for use in treating tumors overexpressing αvβ3 and/or αvβ5 integrins selected from the group consisting of glioblastoma, head and neck cancer, gastroesophageal adenocarcinoma, colorectal cancer, breast cancer, small cell lung cancer, non-small cell lung cancer, malignant melanoma, pancreatic cancer, in particular pancreatic ductal adenocarcinoma, gastric cancer, prostate cancer and brain metastasis, in particular brain metastasis from breast cancer and melanoma, in a human subject, wherein said pharmaceutical composition comprises
[i] a radiolabelled αvβ3 and/or αvβ5 integrins antagonist of formula I:

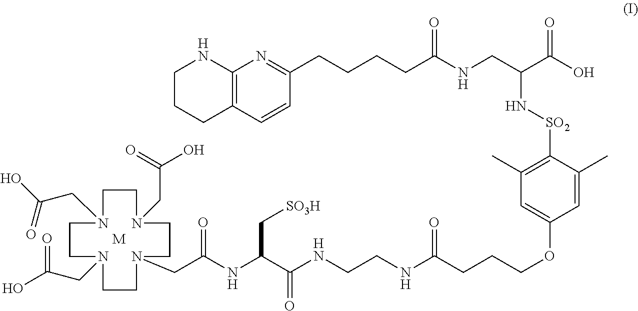

wherein:
M is a radiometal suitable for therapy, preferably 177-Lutetium, and
[ii] one or more pharmaceutically acceptable excipients,
wherein said subject has been selected for the treatment by SPECT/CT or PET/CT or SPECT/MRI, PET/MRI imaging with the same αvβ3 and/or αvβ5 integrins antagonist as defined for the treatment but wherein M is a radiometal suitable for imaging, preferably 68-Gallium, 67-Gallium or 64 Copper, more preferably 68-Gallium.

Similarly, the disclosure relates to a pharmaceutical composition of a radiolabelled αvβ3 and/or αvβ5 integrins antagonist, for use as a imaging agent for SPECT/CT or PET/CT or SPECT/MRI, PET/MRI imaging in determining whether a human subject can be selected for a treatment with radiolabelled αvβ3 and/or αvβ5 integrins antagonist for treating tumors overexpressing αvβ3 and/or αvβ5 integrins selected from the group consisting of glioblastoma, head and neck cancer, gastroesophageal adenocarcinoma, colorectal cancer, breast cancer, small cell lung cancer, non-small cell lung cancer, malignant melanoma, pancreatic cancer, in particular pancreatic ductal adenocarcinoma, gastric cancer, prostate cancer and brain metastasis, in particular brain metastasis from breast cancer and melanoma, wherein said pharmaceutical composition comprises
[i] a radiolabelled αvβ3 and/or αvβ5 integrins antagonist of formula I:

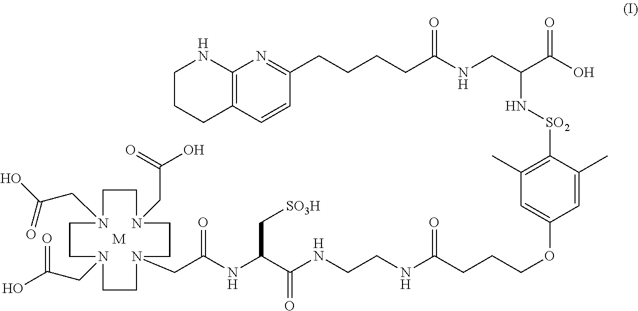

wherein:
M is a radiometal for use as imaging agent in SPECT/CT or PET/CT or SPECT/MRI,
PET/MRI imaging, for example 68-Gallium,
[ii] one or more pharmaceutically acceptable excipients,
wherein said subject is selected for the treatment by evaluating uptake of said radiolabelled αvβ3 and/or αvβ5 integrins antagonist in said tumors overexpressing αvβ3 and/or αvβ5 integrins by SPECT/CT or PET/CT or SPECT/MRI, PET/MRI imaging in said subject.

The disclosure also relates to a method for determining whether a human patient having tumors can be selected for a treatment with a radiolabelled αvβ3 and/or αvβ5 integrins antagonist, said method comprising the steps of:
(i) administering an efficient amount of the pharmaceutical composition of the invention as an imaging agent for imaging the uptake of the radiolabelled αvβ3 and/or αvβ5 integrins antagonist,
(ii) acquiring an image by SPECT/CT, SPECT/MRI, PET/MRI or PET/CT of said patient, and
(iii) comparing with a control image.

### DETAILED DESCRIPTION

### Definitions

As used herein, the term “treat” “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of a tumor, the term “treatment” may refer to the inhibition of the growth of the tumor, or the reduction of the size of the tumor.

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, “MRI” stands for magnetic resonance imaging.

As used herein, “CT” stands for computed tomography.

As used herein, “RLT” stands for radioligand therapy.

As used herein “the Compound” means the compound of formula I of the present disclosure and any form of such Compound, i.e. the Compound as such as well as any of its pharmaceutically acceptable salts, tautomers, pharmaceutically acceptable solvates or pharmaceutically acceptable hydrates thereof.

As used herein, the terms “effective amount” or “therapeutically efficient amount” of a compound refer to an amount of the Compound that will elicit the biological or medical response of a human subject, for example, ameliorate the symptoms, alleviate conditions, slow or delay disease progression, or prevent a disease.

As used herein, “stabilizer(s) against radiolytic degradation” refers to stabilizing agent which protects organic molecules against radiolytic degradation, e.g. when a gamma ray emitted from the radionuclide is cleaving a bond between the atoms of an organic molecules and radicals are forms, those radicals are then scavenged by the stabilizer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”.

As used herein, “sequestering agent” refers to a chelating agent suitable to complex free radionuclide metal ions in the formulation (which are not complexed with the radiolabelled peptide).

As used herein, “pH adjuster”, is chemical that is added to a solution to adjust a pH value of the solution and to thereby achieve a desired performance. Controlling the pH can be performed by adding a pH adjuster to the formulation. Examples of pH adjusters include commonly used acids and bases, buffers and mixtures of acids and bases. For example, bases that can be used include NaOH, KOH, Ca(OH)₂), sodium bicarbonate, sodium acetate, potassium carbonate, and sodium carbonate. Examples of acids that can be used include hydrochloric acid, acetic acid, citric acid, formic acid, fumaric acid, and sulfamic acid.

“Radiochemical purity”: is that percentage of the stated radionuclide that is present in the stated chemical or biological form. Radiochromatography methods, such as HPLC method or instant Thin Layer Chromatography method (iTLC), are commonly accepted methods for determining radiochemical purity in the nuclear pharmacy.

As used herein, “aqueous solution”: a solution of one or more solute in water.

As used herein, “good responder” is a human subject selected from a population which shows statistically better response to a treatment as compared to a randomized population (i.e. which has not been selected by the selection step of the present method), and/or which shows less side effects to a treatment as compared to a randomized population (i.e. which has not been selected by the selection step of the present method).

The term “about” or “ca.” has herein the meaning that the following value may vary for ±20%, preferably ±10%, more preferably ±5%, even more preferably ±2%, even more preferably ±1%.

The Human Subject in Need of Radiolabeled αvβ3 and/or αvβ5 Antagonist Treatment

The terms “patient” and “subject” which are used interchangeably refer to a human being, including for example a subject that has cancer, more specifically, a patient that has αvβ3 and/or αvβ5 overexpressing tumors, as identified for example by ⁶⁸Ga-FF58 PET according to methods described in the present disclosure.

As used herein, the term “cancer” refers to cells having the capacity for autonomous growth, i.e., an abnormal state or condition characterized by rapidly proliferating cell growth.

Hyperproliferative and neoplastic disease states may be categorized as pathologic, i.e., characterizing or constituting a disease state, or may be categorized as non-pathologic, i.e., a deviation from normal but not associated with a disease state. Unless specified otherwise, the term is meant to include all types of cancerous growths or oncogenic processes, metastatic tissues or malignantly transformed cells, tissues, or organs, irrespective of histopathologic type or stage of invasiveness.

As used herein “tumors overexpressing αvβ3 and/or αvβ5 integrins” refers to a tumor which overexpress αvβ3 and/or αvβ5 integrins. The type of the cancer with tumors overexpressing αvβ3 and/or αvβ5 integrins includes without limitation glioblastoma, head and neck cancer, gastroesophageal adenocarcinoma, colorectal cancer, breast cancer, small cell lung cancer, non-small cell lung cancer, malignant melanoma, gastric cancer, pancreatic cancer, in particular pancreatic ductal adenocarcinoma, prostate cancer and brain metastasis, in particular brain metastasis from breast cancer and melanoma.

The Compound is preferably used for the suppression of solid cancer, preferably selected from head and neck cancer, gastroesophageal adenocarcinoma, colorectal cancer, renal cancer, breast cancer such as invasive ductal breast cancer, small-cell lung cancer, non-small cell lung cancer, glioblastoma, malignant melanoma, gastric cancer, pancreatic cancer, in particular pancreatic ductal adenocarcinoma, prostate cancer, or is preferably used for the suppression of metastasis preferably selected from brain metastasis from breast cancer and melanoma, preferably melanoma.

Radiolabeled αvβ3 and/or αvβ5 Antagonist for Use According to the Disclosure

The present disclosure relates to a theragnostic approach for treating tumors overexpressing αvβ3 and/or αvβ5 integrins in a subject in need thereof.

The theragnostic approach advantageously comprises a first imaging step using a radiolabelled αvβ3 and/or αvβ5 antagonist for selecting patient with tumors overexpressing αvβ3 and/or αvβ5 integrins suitable for the treatment step, and a second treatment step for treating the patient with the corresponding radiolabelled αvβ3 and/or αvβ5 antagonist. Hence, in specific embodiments, the same αvβ3 and/or αvβ5 antagonist compound is used for the imaging step for selecting the patient suitable for the treatment and for the treatment step, but the radiometal is different, one being suitable for use as imaging agent for imaging, and the other for use as therapeutic agent for nuclear therapy.

As used herein an αvβ3 and/or αvβ5 antagonist refers to a compound comprising at least a small peptidic moiety which binds to the αvβ3 and αvβ5 integrins with high affinity and specificity, and which is not internalized upon receptor binding and antagonizes the effect of the natural ligand at certain concentration.

More specifically, the αvβ3 and/or αvβ5 antagonist refers to a compound of the following formula (I):

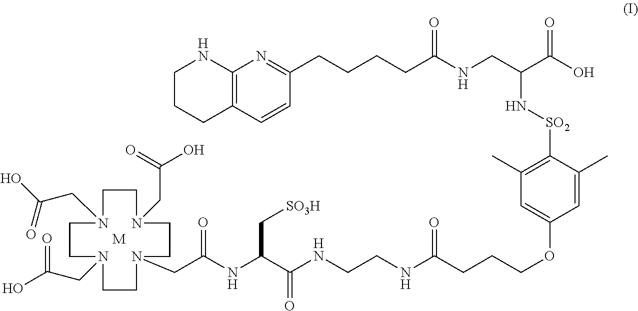

or any pharmaceutically acceptable salts, hereafter referred as the Compound.

The Compound further contains a DOTA metal-chelator in its structure which allows for radiolabelling with different radionuclides M such as 68-Gallium (for imaging), 177Lutetium (for RLT) and other relevant radionuclides, which makes the theragnostic use of such compound possible.

In addition, the Compound has favorable pharmacokinetic (PK) and plasma stability for use in the detection of tumors.

Further details of the Compound, its manufacturing method and uses are disclosed in particular in EP3050878A1 or US2018008583 patent publications, which disclosures are incorporated herein by reference.

In an embodiment, M is a radiometal which can be selected from, ¹¹¹In, ^{133m}In, ^{99m}Tc, ^{94m}Tc, ⁶⁷Ga, ⁶⁶Ga, ⁶⁸Ga, ⁵²Fe, ¹⁶⁹Er, ⁷²As, ⁹⁷Ru, ²⁰³Pb, ²¹²Pb, ⁶²Cu, ⁶⁴Cu, ⁶⁷Cu, ¹⁸⁶Re, ¹⁸⁸Re, ⁸⁶Y, ⁹⁰Y, ⁵¹Cr, ^{52m}Mn, ¹⁵⁷Gd, ¹⁷⁷Lu, ¹⁶¹Tb, ¹⁶⁹Yb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn ²¹³Bi, ²¹²Bi, ¹⁴²Pr, ¹⁴³Pr, ¹⁹⁸Au, ¹⁹⁹Au, ⁸⁹Zr, ²²⁵Ac, ⁴³Sc, ⁴⁴Sc, ⁴⁷Sc, and ⁵⁵Co.

Typical radiometal suitable for use as imaging agent in SPECT/CT or PET/CT or SPECT/MRI, PET/MRI imaging can be selected from ¹¹¹In, ^{133m}In ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁵⁷Gd, ¹⁶⁹Yb, ¹⁷²Tm, ^{117m}Sn, ⁸⁹Zr, ⁴³Sc, ⁴⁴Sc, ⁵⁵Co. According to a preferred embodiment for use in SPECT/CT or PET/CT or SPECT/MRI, PET/MRI imaging, M is ⁶⁸Ga. In this case, the radiolabeled αvβ3/αvβ5 antagonist can be used as imaging agent for PET/CT or PET/MRI or SPECT/CT or SPECT/MRI imaging for the patient selection step.

Typical radiometal for use in the treatment step for RLT includes the following: ¹⁶⁹Er, ²¹²Pb, ⁶⁴Cu, ⁶⁷Cu, ¹⁸⁶Re, ¹⁸⁸Re, ⁹⁰Y, ¹⁷⁷Lu, ¹⁶¹Tb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹²¹Sn, ²¹³Bi, ²¹²Bi, ¹⁴²Pr, ¹⁴³Pr, ¹⁹⁸Au, ¹⁹⁹Au, ²²⁵Ac, ⁴⁷Sc. According to a specific embodiment radiometal M for use in the treatment step for RLT is ¹⁷⁷Lu. The subject is selected for the treatment by evaluating the αvβ3/αvβ5 antagonist uptake in the tumors overexpressing αvβ3/αvβ5 integrins as determined by PET/MRI or PET/CT or SPECT/CT or SPECT/MRI imaging in said subject.

Synthesis of the αvβ3 and/or αvβ5 Antagonist Formula (II)

The cold compound, i.e. the non-radiolabeled αvβ3 and/or αvβ5 antagonist, is of formula (II):

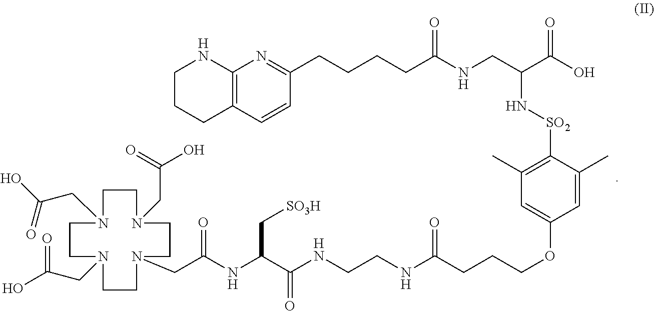 [SMILES: Cc1cc(OCCCC(=O)NCCNC(=O)[C@H](CS(=O)(=O)O)NC(=O)CN2CCN(CC(=O)O)CCN(CC(=O)O)CCN(CC(=O)O)CC2)cc(C)c1S(=O)(=O)NC(CNC(=O)CCCCc1ccc2c(n1)NCCC2)C(=O)O]

The cold compound of formula (II) can be synthesized using the methods disclosed in the patent publications EP3050878A1 or US2018008583.

The Pharmaceutical Composition

The disclosure also relates to pharmaceutical composition of a radiolabelled αvβ3 and/or αvβ5 integrins antagonist which comprises:
[i] a radiolabelled αvβ3 and/or αvβ5 integrins antagonist of formula I:

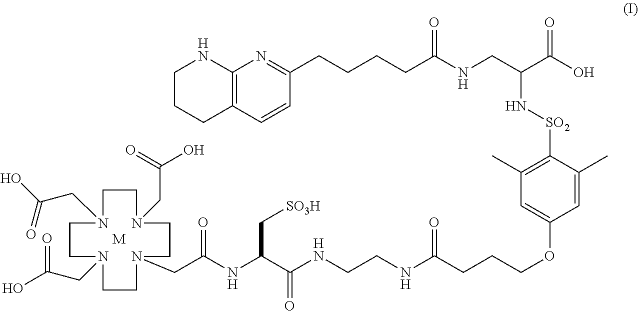

wherein:
M is a radiometal suitable for therapy, preferably 177-Lutetium, and
[ii] one or more pharmaceutically acceptable excipients. The Compound with M being ¹⁷⁷Lu is hereafter referred as the [¹⁷⁷Lu]-Compound.

In an embodiment, the [¹⁷⁷Lu]-Compound can be present in a concentration providing a volumetric radioactivity of at least 10 mCi/mL (370 MBq/mL), preferably at least 15 mCi/mL (555 MBq/mL), more preferably at least 20 mCi/mL (740 MBq/mL) in said pharmaceutical composition. The [¹⁷⁷Lu]-Compound can be present in a concentration providing a volumetric radioactivity comprised between 70 mCi/mL (2590 MBq/mL) and 400 mCi/mL (1.48×10⁴ MBq/mL), preferably between 90 mCi/mL (3330 MBq/mL) and 385 mCi/mL (1.42×10⁴ MBq/mL), for example, at a concentration of about 90 mCi/mL (3330 MBq/mL), or 99 mCi/mL (3663 MBq/mL), or 168 mCi/mL (6216 MBq/mL), or 184 mCi/mL (6808 MBq/mL), or 361 mCi/mL (1.34×10⁴ MBq/mL) in said pharmaceutical composition.

In an embodiment, the specific activity of said pharmaceutical composition comprising the [¹⁷⁷Lu]-Compound as therapeutic agent expressed as GBq/Total peptide is between 1 GBq/mg and 75 GBq/mg, preferably between 18.5 GBq/mg and 55 GBq/mg, more preferably between 30 GBq/mg and 44 GBq/mg, even more preferably between 34 GBq/mg and 40 GBq/mg, preferably about 37 GBq/μmol (1 Ci/mg).

In an embodiment, the molar ratio between the Compound and the radionuclide can be at least 1.3, preferably between 1.3 and 1.8, more preferably about 1.5.

The one or more pharmaceutically acceptable excipients can be any of those conventionally used, and is limited only by physico-chemical considerations, such as solubility and lack of reactivity with the active compound(s).

In particular, the one or more pharmaceutically acceptable excipients can be selected from stabilizers against radiolytic degradation, solvents, sequestering agents, pH adjusters and mixtures thereof.

In a first aspect, the present disclosure relates to a pharmaceutical composition comprising the [¹⁷⁷Lu]-Compound as described herein, and at least two stabilizers against radiolytic degradation. In an embodiment, said at least two stabilizers can be selected from gentisic acid (2,5-dihydroxybenzoic acid) or salts thereof, ascorbic acid (L-ascorbic acid, vitamin C) or salts thereof (e.g. sodium ascorbate), methionine, histidine, melatonine, and Se-methionine, preferably selected from gentisic acid or salts thereof and ascorbic acid or salts thereof. More preferably, said at least two stabilizers are gentisic acid and sodium ascorbate.

In particular, the inventors unexpectedly found that adding both gentisic acid and sodium ascorbate in specific amounts in a pharmaceutical composition of the [¹⁷⁷Lu]-Compound enables a radiochemical purity of said composition over 95% after 72 hours after synthesis, preferably 5 days after synthesis.

In an embodiment, the ratio between gentisic acid and sodium ascorbate, is between 1:100 and 1:200, preferably between 1:110 and 1:150, more preferably between 1:120 and 1:135, even more preferably about 1:128.

In an embodiment, said gentisic acid or salts thereof, preferably gentisic acid, can be present in a concentration of least 0.30 mg/mL, preferably at least 0.35 mg/mL, and more preferably at least 0.38 mg/mL, typically between 0.30 mg/mL and 0.40 mg/mL, preferably between 0.38 mg/mL and 0.40 mg/mL, more preferably about 0.39 mg/mL.

In an embodiment, said ascorbic acid or salts thereof, preferably sodium ascorbate, can be present in a concentration of at least 40 mg/mL, preferably at least 45 mg/mL, typically between 40 mg/mL and 75 mg/mL, preferably between 45 mg/mL and 65 mg/mL, more preferably about 50 mg/mL.

In an embodiment, said gentisic acid or salts thereof is present in a concentration between 0.30 mg/mL and 0.40 mg/mL, preferably between 0.38 mg/mL and 0.40 mg/mL, and ascorbic acid or salts thereof can be present at a concentration between 40 mg/mL and 75 mg/mL, preferably between 45 mg/mL and 65 mg/mL.

In an embodiment, the radiopharmaceutical composition comprises, as radiostabilizers, both gentisic acid and sodium ascorbate, at the respective concentrations of 0.39 mg/mL and 50 mg/mL.

In an embodiment, the pharmaceutical composition has radiochemical purity higher than 95% up to 72 hours and 120 hours, in particular up to 5 days, preferably equal to or higher than 97% up to 72 hours and equal to or higher than 96.5% up to 120 hours.

In an embodiment the pH of the pharmaceutical composition as described herein can be between 4.5 and 7, preferably between 5.5 and 6.5, more preferably about 6.

In a second aspect, the present disclosure relates to a pharmaceutical composition comprising the [¹⁷⁷Lu]-Compound as described herein, at least two stabilizers against radiolytic degradation and at least one other pharmaceutically acceptable excipient. The pharmaceutically acceptable excipient can be any of those conventionally used, and is limited only by physico-chemical considerations, such as solubility and lack of reactivity with the active compound(s). In particular, the at least other pharmaceutically acceptable excipient can be selected from solvent, sequestering agent, pH adjuster and mixtures thereof.

In an embodiment said solvent is water for injection and/or saline solution, preferably water for injection.

In an embodiment said sequestering agent is diethylentriaminepentaacetic acid (DTPA) or a salt thereof. In an embodiment said DTPA is present at a concentration between 50 and 200 μg/mL, preferably between 75 and 150 μg/mL, more preferably about 100 μg/mL.

In an embodiment said pH adjuster is acetic acid and/or sodium acetate, preferably acetic acid and sodium acetate. In an embodiment said acetic acid is present at a concentration between 0.20 mg/mL and 0.40 mg/mL, preferably about 0.30 mg/mL and said sodium acetate is present at a concentration between 0.30 mg/mL and 0.50 mg/mL, preferably about 0.41 mg/mL.

In another aspect of the disclosure, the pharmaceutical composition is produced at commercial scale manufacturing, in particular is produced at a batch size of at least 1 Ci (37 GBq), preferably 2 Ci (74 GBq).

In another aspect of the disclosure, the pharmaceutical composition is for commercial use.

Radiolabelling of the Cold αvβ3 and/or αvβ5 Antagonist of Formula (II)

In general, a method for labelling the αvβ3 and/or αvβ5 antagonist of formula (II)_with a radiometal M as disclosed, comprises the steps of:
i. providing said αvβ3 and/or αvβ5 antagonist of formula (II) in a vial

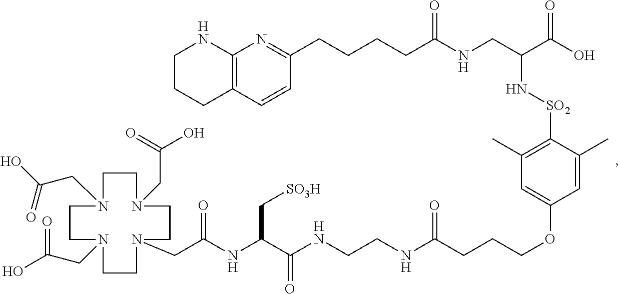 [SMILES: Cc1cc(OCCCC(=O)NCCNC(=O)[C@H](CS(=O)(=O)O)NC(=O)CN2CCN(CC(=O)O)CCN(CC(=O)O)CCN(CC(=O)O)CC2)cc(C)c1S(=O)(=O)NC(CNC(=O)CCCCc1ccc2c(n1)NCCC2)C(=O)O]

ii. adding a solution of said radiometal into said vial, thereby obtaining a solution of said αvβ3 and/or αvβ5 antagonist of formula (II) with said radioactive isotope, and
iii. mixing the solution obtained in ii, and incubating it for a sufficient period of time for obtaining said αvβ3 and/or αvβ5 antagonist labelled with said radioactive isotope of formula (I).

The [¹⁷⁷Lu]-Compound can be manufactured both automatically, for example by using the MiniAIO synthesizer or other synthesizers known in the art for automated synthesis, and manually.

The synthesis can be conducted by firstly transferring the ¹⁷⁷LuCl₃ into the reactor. Then a reaction buffer is added to a vial previously containing ¹⁷⁷LuCl3. The reaction buffer comprises a solvent, preferably water for injection, a buffer preferably acetate buffer and a stabilizer against radiolytic degradation, preferably gentisic acid. Then the reaction buffer solution can be transferred in a reactor. The Compound of formula (II) can be in a second vial which is added to the reactor containing the reaction buffer and ¹⁷⁷LuCl3 previously obtained in the first vial. The labelling can be conducted at 95° C. for at least 5 minutes. And finally, at the end of labelling, the [¹⁷⁷Lu]-Compound obtained can be transferred to a mother vial. The mother vial can be filtered and diluted in order to obtain a volumetric activity of 21 mCi/mL.

The radiometal used in this method is as disclosed herein.

The Pharmaceutical Composition for Use as the Imaging Agent in the Imaging Step

The pharmaceutical composition for use as imaging agent in the imaging step comprises the radiolabeled αvβ3/αvβ5 antagonist as described herein, i.e. the Compound and one or more pharmaceutically acceptable excipients.

More specifically said pharmaceutical composition comprises
[i] a radiolabelled αvβ3 and/or αvβ5 integrins antagonist of formula I:

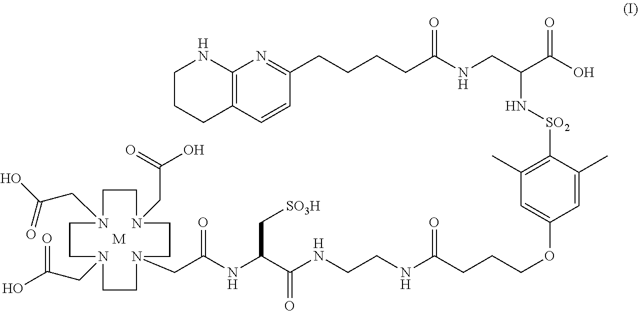

or any pharmaceutically acceptable salts,
wherein:
M is a radiometal for use as imaging agent in SPECT/CT or PET/CT or SPECT/MRI, PET/MRI imaging, and
[ii] one or more pharmaceutically acceptable excipients.

Radiometal suitable for use as imaging agent in SPECT/CT or PET/CT or SPECT/MRI, PET/MRI imaging can be selected from ¹¹¹In, ^{133m}In ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁵⁷Gd, ¹⁶⁹Yb, ¹⁷²Tm, ^{117m}Sn, ⁸⁹Zr, ⁴³Sc, ⁴⁴Sc, ⁵⁵Co. According to a preferred embodiment, M is ⁶⁸Ga. The Compound with M being ⁶⁸Ga is hereafter referred as the [⁶⁸Ga]-Compound.

In an embodiment, the [⁶⁸Ga]-Compound can be present in a concentration providing a volumetric radioactivity of at least 20 MBq/mL, preferably at least 50 MBq/mL in said pharmaceutical composition. The Compound can be present in a concentration providing a volumetric radioactivity comprised between 20 MBq/mL and 1000 MBq/mL, preferably between 30 MBq/mL and 600 MBq/mL, for example, at a concentration of about 200 MBq/mL in said pharmaceutical composition.

In an embodiment, the specific activity of said pharmaceutical composition comprising the [68Ga]-Compound as imaging agent expressed as GBq/Total peptide is between 3 GBq/μmol and 100 GBq/μmol, preferably between 4 GBq/μmol and 90 GBq/μmol, more preferably between 5 GBq/μmol and 80 GBq/μmol, even more preferably between 6 GBq/μmol and 70 GBq/μmol, preferably about 50 GBq/μmol.

The one or more pharmaceutically acceptable excipients can be any of those conventionally used, and is limited only by physico-chemical considerations, such as solubility and lack of reactivity with the active compound(s).

In particular, the one or more pharmaceutically acceptable excipients can be selected from stabilizer(s) against radiolytic degradation, buffers, sequestering agents and mixtures thereof.

Due to the radioactive nature of the Compound, a decay of the radionuclide activity occurs, which then becomes an inherent property of the radiopharmaceutical. Consequently, the specific activity, total radioactivity and radioconcentration of the Compound changes over the time.

Suitable stabilizer(s) against radiolytic degradation, may be used to ensure high stability, at least 95%, 96%, 97%, 98%, 99% or 100% chemical stability with respect to the radiochemical purity radionuclide complex, e.g the Compound, after 24 hours at 25° C., even if this Compound is a sensitive peptide molecule.

In a preferred embodiment, the pharmaceutical composition for use as imaging agent in the imaging step comprises a stabilizer against radiolytic degradation, preferably, gentisic acid.

According to an embodiment, the pharmaceutical composition for use as imaging agent in the imaging step is an aqueous solution, for example an injectable formulation. According to a particular embodiment, the pharmaceutical composition of the [⁶⁸Ga]-Compound is a solution for bolus injection.

The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and SHP Handbook on Injectable Drugs, Trissel, 15th ed., pages 622-630 (2009)).

The Pharmaceutical Composition for Use as the Therapeutic Agent in the Treatment Step

The pharmaceutical composition for use as the therapeutic agent in the treatment step comprises the radiolabeled αvβ3/αvβ5 antagonist as described herein, i.e. the Compound and one or more pharmaceutically acceptable excipients.

More specifically said pharmaceutical composition comprises
[i] a radiolabelled αvβ3 and/or αvβ5 integrins antagonist of formula I:

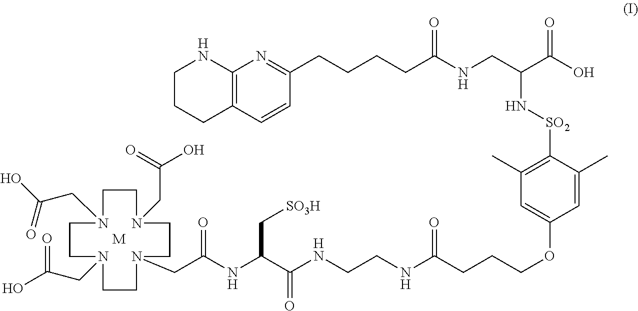

any pharmaceutically acceptable salts,
wherein:
M is a radiometal suitable for therapy, and
[ii] one or more pharmaceutically acceptable excipients.

Radiometal suitable for RLT includes the following: ¹⁶⁹Er, ²¹²Pb, ⁶⁴Cu, ⁶⁷Cu, ¹⁸⁶Re, ¹⁸⁸Re, ⁹⁰Y, ¹⁷⁷Lu, ¹⁶¹Tb, ¹⁷⁵Yb, ¹⁰⁵Rh, ¹⁶⁶Dy, ¹⁶⁶Ho, ¹⁵³Sm, ¹⁴⁹Pm, ¹⁵¹Pm, ¹²¹Sn, ²¹³Bi, ²¹²Bi, ¹⁴²Pr, ¹⁴³Pr, ¹⁹⁸Au, ¹⁹⁹Au, ²²⁵Ac, ⁴⁷Sc. According to a preferred embodiment, M is ¹⁷⁷Lu. The Compound with M being ¹⁷⁷Lu is hereafter referred as the [¹⁷⁷Lu]-Compound.

The one or more pharmaceutically acceptable excipients can be any of those conventionally used, and is limited only by physico-chemical considerations, such as solubility and lack of reactivity with the active compound(s).

In particular, the one or more pharmaceutically acceptable excipients can be selected from stabilizer(s) against radiolytic degradation, buffers, sequestering agents and mixtures thereof.

According to an embodiment, the pharmaceutical composition for use as the therapeutic agent in the treatment step is an aqueous solution, for example an injectable formulation. According to a particular embodiment, the pharmaceutical composition of the [¹⁷⁷Lu]-Compound is a solution for infusion.

The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and SHP Handbook on Injectable Drugs, Trissel, 15th ed., pages 622-630 (2009)).

Methods for Selecting a Subject for αvβ3/αvβ5 Antagonist Treatment

The disclosure also relates to methods for determining whether a human patient having tumor lesions overexpressing αvβ3/αvβ5 integrins can be selected for a treatment of said tumor lesions with a radiolabelled αvβ3 and/or αvβ5 integrins antagonist, said method comprising the steps of:
(i) administering an efficient amount of the pharmaceutical composition for use as a imaging agent of the present disclosure for imaging the uptake of the radiolabelled αvβ3 and/or αvβ5 integrins antagonist in said subject,
(ii) acquiring an image by PET/MRI or PET/CT or SPECT/CT or SPECT/MRI of said patient.
(iii) determining the uptake of the radiolabelled αvβ3 and/or αvβ5 integrins antagonist based on the image acquired at step (ii) in said tumor lesions.

In a specific embodiment of the method, said patient is a patient having tumors selected from the group consisting of glioblastoma, head and neck cancer, colorectal cancer, breast cancer, small cell lung cancer, non-small cell lung cancer, malignant melanoma, gastric cancer, pancreatic cancer, prostate cancer and brain metastasis, in particular brain metastasis from breast cancer and melanoma. In a more specific embodiment, the radiolabelled αvβ3 and/or αvβ5 antagonist is the [⁶⁸Ga]-Compound.

In an embodiment, the pharmaceutical composition for use as an imaging agent is the pharmaceutical composition comprising the [⁶⁸Ga]-Compound.

In an embodiment, the pharmaceutical composition the [⁶⁸Ga]-Compound as imaging agent is administered to said subject by intravenous injection, preferably for bolus injection.

In another embodiment, the pharmaceutical composition comprising the [⁶⁸Ga]-Compound as imaging agent is administered between 6 hours and 15 minutes, preferably between 3 hours and 25 minutes, before step (ii) of PET/CT or PET/MRI or SPECT/CT or SPECT/MRI imaging.

The dose of the pharmaceutical composition comprising the [⁶⁸Ga]-Compound as imaging agent in the present method differs depending on the age, sex, and symptoms of a patient, an administration route, the number of doses, and a dosage form. In general, the radioactive dose of the pharmaceutical composition comprising the [⁶⁸Ga]-Compound can be selected, for example, within the range of between 50 MBq and 350 MBq per injection, preferably between 150 MBq and 250 MBq. The dose is calculated based on the subject's body weight at a dose of about 3 MBq/kg for one dose. In an embodiment, the human patient only receives one injection of the [⁶⁸Ga]-Compound.

Images of patient's body are then acquired by PET/MRI or PET/CT or SPECT/CT or SPECT/MRI imaging and the images are compared with a control image to identify whether the lesions identified by conventional imaging, for example by MRI, CT, are also identified by [⁶⁸Ga]-Compound uptake.

αvβ3 and/or αvβ5 expressing tumors may be advantageously detected by evaluating the uptake of the pharmaceutical composition for use as imaging agent of the present disclosure by PET/MRI or PET/CT or SPECT/CT or SPECT/MRI imaging after injection of said pharmaceutical composition. The objective of the above method is therefore to select subject with αvβ3 and/or αvβ5 overexpressing tumors, and who may be good responders to a RLT with the pharmaceutical composition for use as therapeutic agent.

In a specific embodiment of the method, the patients selected for said treatment are the patients having at least 10%, preferably more than 20%, preferably more than 30%, preferably more than 40%, preferably more than 50%, preferably more than 60%, preferably more than 70%, preferably more than 80%, preferably more than 90%, preferably between 90% and 95% of the lesions detected by conventional imaging which also exhibits [68Ga]-αvβ3 and/or αvβ5 antagonist uptake as determined by PET/MRI or PET/CT or SPECT/CT or SPECT/MRI with said [68Ga]-αvβ3 and/or αvβ5 antagonist.

In specific embodiment, the term “lesion” refers to measurable tumor lesions as defined in the published RECIST document available at http://www.eortc.be. Typically, measurable tumor lesions are lesions with a minimum size (the longest diameter in the plane of measurement is to be recorded) of:
10 mm by CT scan (CT scan slice thickness no greater than 5 mm);
10 mm caliper measurement by clinical exam (lesions which cannot be accurately measured with calipers should be recorded as non-measurable);
20 mm by chest X-ray.

Non-measurable are all other lesions, including small lesions (longest diameter <10 mm or pathological lymph nodes with ≥10 to <15 mm short axis) as well as truly non-measurable lesions. Lesions considered truly non-measurable include leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

All measurements should be recorded in metric notation, using calipers if clinically assessed. All baseline evaluations should be performed as close as possible to the treatment start.

In specific embodiments, a lesion identified by conventional imaging will be considered a tumor lesion overexpressing αvβ3 and/or αvβ5 integrins for the purpose of the present patient selection method, if [68Ga]-Compound uptake in the lesion is equal or superior (visual assessment) to the spleen uptake.

In other specific embodiments, a lesion is determined as overexpressing αvβ3 and/or αvβ5 integrins for [68Ga]-Compound uptake by determining the ratio between the mean SUV of each region of interest drawn (potential lesions) to the mean SUV of the aorta the ratio between the mean SUV of each region of interest drawn (potential lesions) to the mean SUV of the aorta (SUVr). Typically, a lesion is determined as overexpressing αvβ3 and/or αvβ5 integrins if the SUVr values are above.

The disclosure relates to a pharmaceutical composition of a radiolabeled αvβ3 and/or αvβ5 integrins antagonist, for use as a imaging agent for PET/CT or PET/MRI or SPECT/CT or SPECT/MRI imaging in determining whether a human subject can be selected for a treatment with radiolabeled αvβ3 and/or αvβ5 integrins antagonist for treating tumors overexpressing αvβ3 and/or αvβ5 integrins, said tumors being selected from the group consisting of glioblastoma, head and neck cancer, colorectal cancer, breast cancer, small cell lung cancer, non-small cell lung cancer, malignant melanoma, gastric cancer, pancreatic cancer, prostate cancer and brain metastasis, in particular brain metastasis from breast cancer and melanoma, wherein said subject is selected for the treatment by evaluating uptake of said radiolabeled αvβ3 and/or αvβ5 integrins in tumors overexpressing αvβ3 and/or αvβ5 integrins by PET/CT or PET/MRI or SPECT/CT or SPECT/MRI imaging in said subject.

In certain embodiments, the present method then further comprises a step (iv) of treating tumors overexpressing αvβ3 and/or αvβ5 integrins in said patient selected for such treatment, said step comprising administering a therapeutically efficient amount of the pharmaceutical composition for use as therapeutic agent in the treatment step, which pharmaceutical compositions comprises as the active ingredient, the same αvβ3 and/or αvβ5 integrins antagonist used in the selection step (i) but having a radiometal suitable for RLT. Advantageously, the radiolabeled αvβ3 and/or αvβ5 integrins antagonist for use as therapeutic agent in the treatment step is [¹⁷⁷Lu]-Compound.

In an embodiment, a therapeutically efficient amount of [¹⁷⁷Lu]-Compound is administered by intravenous injection, for example as a solution for infusion.

In an embodiment, the pharmaceutical composition comprising [¹⁷⁷Lu]-Compound can be administered at a radioactive dose of between 1.85 GBq and 18.5 GBq (50-500 mCi) per injection. The dose is calculated based on the ascending dose and dosimetry.

In specific embodiments, a therapeutically efficient amount of the [¹⁷⁷Lu]-Compound is administered to said subject 1 to 8 times per treatment, for example 2 to 4 times.

In an embodiment, the therapeutically efficient amount of the [¹⁷⁷Lu]-Compound is administered at least two weeks after step (i) of administering the imaging agent in the imaging step.

In certain aspects, the administration of the pharmaceutical composition which comprises [¹⁷⁷Lu]-Compound at the treatment step can inhibit, delay, and/or reduce tumor growth in the subject. In certain aspects, the growth of the tumor is delayed by at least 5%, 10%, 20%, 30%, 40%, 50%, 60%, 70% or 80% in comparison to an untreated control subject.

In certain aspects, the growth of the tumor is delayed by at least 5%, 10%, 20%, 30%, 40%, 50%, 60%, 70% or 80% in comparison to an untreated control subject. In certain aspects, the growth of the tumor is delayed by at least 5%, 10%, 20%, 30%, 40%, 50%, 60%, 70% or 80% in comparison to the predicted growth of the tumor without the treatment. In certain aspects, the growth of the tumor is delayed by at least 50% in comparison to the predicted growth of the tumor without the treatment.

In certain aspects, the administration of the pharmaceutical composition which comprises [¹⁷⁷Lu]-Compound at the treatment step can increase the length of survival of the subject. In certain aspects, the increase in survival is in comparison to an untreated control subject. In certain aspects, the increase in survival is in comparison to the predicted length of survival of the subject without the treatment. In certain aspects, the length of survival is increased by at least 3 times, 4 times, or 5 times the length in comparison to an untreated control subject. In certain aspects, the length of survival is increased by at least 4 times the length in comparison to an untreated control subject.

In certain aspects, the length of survival is increased by at least 3 times, 4 times, or 5 times the length in comparison to the predicted length of survival of the subject without the treatment. In certain aspects, the length of survival is increased by at least 4 times the length in comparison to the predicted length of survival of the subject without the treatment.

In certain aspects, the length of survival is increased by at least one week, two weeks, one month, two months, three months, six months, one year, two years, or three years in comparison to an untreated control subject.

In certain aspects, the length of survival is increased by at least one month, two months, or three months in comparison to an untreated control subject. In certain aspects, the length of survival is increased by at least one week, two weeks, one month, two months, three months, six months, one year, two years, or three years in comparison to the predicted length of survival of the subject without the treatment. In certain aspects, the length of survival is increased by at least one month, two months, or three months in comparison to the predicted length of survival of the subject without the treatment.

In an embodiment, the pharmaceutical composition which comprises [¹⁷⁷Lu]-Compound of the present disclosure may be used in combination with one or more anticancer agent. Examples of such anticancer agent include without limitation alkylating agents, antimetabolites, microtubule inhibitors, anticancer antibiotics, topoisomerase inhibitors, platinum preparations, molecular targeting drugs, hormones, immuno-oncology and biologics.

Typical alkylating agents include nitrogen mustard anticancer agents such as cyclophosphamide, nitrosourea anti-cancer agents such as ranimustine, and dacarbazine. Examples of the antimetabolites include 5-FU, UFT, carmofur, capecitabine, tegafur, TS-1, gemcitabine, and cytarabine. Examples of the microtubule inhibitors include alkaloid anticancer agents such as vincristine, and taxane anticancer agents such as docetaxel and paclitaxel. Examples of the anticancer antibiotics include mitomycin C, doxorubicin, epirubicin, daunorubicin, and bleomycin. Examples of the topoisomerase inhibitors include irinotecan and nogitecan having a topoisomerase I inhibitory effect, and etoposide having a topoisomerase II inhibitory effect. Examples of the platinum preparations include cisplatin, Paraplatin, nedaplatin, and oxaliplatin. Examples of the molecular targeting drugs include trastuzumab, rituximab, imatinib, gefitinib, erlotinib, bevacizumab, cetuximab, panitumumab, bortezomib, sunitinib, sorafenib, crizotinib, and regorafenib. Examples of the hormones include dexamethasone, finasteride, and tamoxifen. Examples of the biologics include interferons α, 6, and γ and interleukin 2.

In another embodiment, the pharmaceutical composition which comprises [¹⁷⁷Lu]-Compound may be used in combination with a cancer therapy and can be used in combination with surgical operation as well as radiotherapy (including gamma knife therapy, cyberknife therapy, boron neutron capture therapy, proton radiation therapy, and heavy particle radiotherapy), MR-guided focused ultrasound surgery, cryotherapy, radiofrequency ablation, percutaneous ethanol injection therapy, arterial embolization, or the like.

### Embodiments

1. A pharmaceutical composition of a radiolabelled αvβ3 and/or αvβ5 integrins antagonist, for use in treating tumors overexpressing αvβ3 and/or αvβ5 integrins selected from the group consisting of glioblastoma, head and neck cancer, colorectal cancer, breast cancer, small cell lung cancer, non-small cell lung cancer, malignant melanoma, gastric cancer, pancreatic cancer, prostate cancer and brain metastasis, in particular brain metastasis from breast cancer and melanoma, in a human subject, wherein said pharmaceutical composition comprises
   [i] a radiolabelled αvβ3 and/or αvβ5 integrins antagonist of formula I:

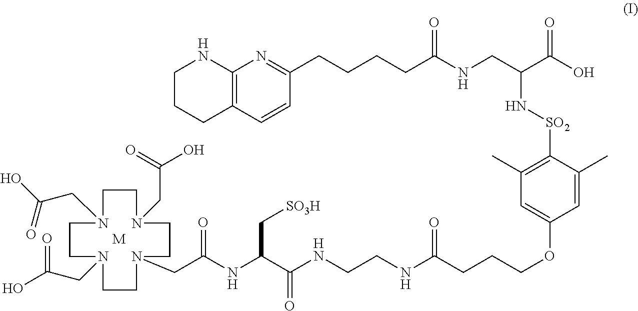

wherein:
M is a radiometal suitable for therapy, preferably 177-Lutetium, and
[ii] one or more pharmaceutically acceptable excipients,
wherein said subject has been selected for the treatment by SPECT/CT or PET/CT or SPECT/MRI, PET/MRI imaging with the same αvβ3 and/or αvβ5 integrins antagonist as defined for the treatment but wherein M is a radiometal suitable for imaging, preferably 68-Gallium, 67-Gallium or 64-Copper, more preferably 68-Gallium.
2. The pharmaceutical composition for use according to embodiment 1, wherein tumors overexpressing αvβ3 and/or αvβ5 integrins are selected from the group consisting of glioblastoma, malignant melanoma, brain metastasis from breast cancer and melanoma, preferably malignant melanoma.
3. The pharmaceutical composition for use according to embodiment 1 or 2, wherein the pharmaceutical composition is suitable for injection, preferably for infusion.
4. The pharmaceutical composition for use according to any of embodiments 1 to 3, wherein, a subject has been selected for the treatment by evaluating [⁶⁸Ga]-labeled αvβ3 and/or αvβ5 integrins antagonist uptake in the lesions as determined by PET/MRI or PET/CT or SPECT/CT or SPECT/MRI imaging in said subject.
5. A pharmaceutical composition of a radiolabelled αvβ3 and/or αvβ5 integrins antagonist, for use as a imaging agent for PET/CT or PET/MRI or SPECT/CT or SPECT/MRI imaging in determining whether a subject can be selected for a treatment with radiolabelled αvβ3 and/or αvβ5 integrins antagonist for treating tumors overexpressing αvβ3 and/or αvβ5 integrins selected from the group consisting of glioblastoma, head and neck cancer, gastroesophageal adenocarcinoma, colorectal cancer, breast cancer, small cell lung cancer, non-small cell lung cancer, malignant melanoma, gastric cancer, pancreatic cancer, in particular pancreatic ductal adenocarcinoma, prostate cancer and brain metastasis, in particular brain metastasis from breast cancer and melanoma, wherein said pharmaceutical composition comprises
   [i] a radiolabelled αvβ3 and/or αvβ5 integrins antagonist of formula I:

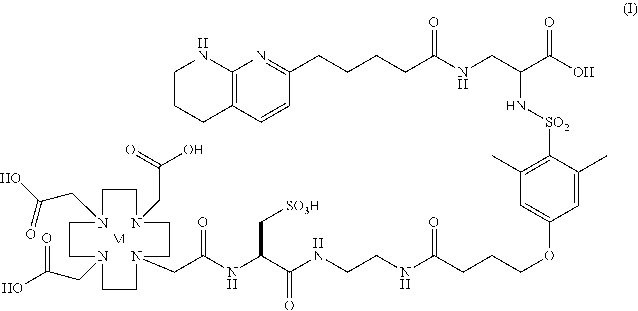

wherein:
M is a radiometal for use as imaging agent in SPECT/CT or PET/CT or SPECT/MRI, PET/MRI imaging, for example 68-Gallium, and
[ii] one or more pharmaceutically acceptable excipients,
wherein said subject is selected for the treatment by evaluating uptake of said radiolabelled αvβ3 and/or αvβ5 integrins antagonist in said tumors overexpressing αvβ3 and/or αvβ5 integrins by PET/CT or PET/MRI or SPECT/CT or SPECT/MRI imaging in said subject.
6. The pharmaceutical composition for use according to embodiment 5, wherein tumors overexpressing αvβ3 and/or αvβ5 integrins are selected from the group consisting of glioblastoma, malignant melanoma, brain metastasis from breast cancer and melanoma, preferably melanoma.
7. A method for determining whether a human patient having tumors can be selected for a treatment with a radiolabelled αvβ3 and/or αvβ5 integrins antagonist, said method comprising the steps of:
   (i) administering an efficient amount of the pharmaceutical composition of embodiments 5 or 6 as an imaging agent for imaging the uptake of the radiolabelled αvβ3 and/or αvβ5 integrins antagonist,
   (ii) acquiring an image by PET/MRI or PET/CT or SPECT/CT or SPECT/MRI of said patient, and
   (iii) comparing with a control image.
8. The method of embodiment 7, further comprising a step of treating tumors overexpressing αvβ3 and/or αvβ5 integrins by administering a therapeutically efficient amount of the pharmaceutical composition of embodiments 1 to 3.
9. The method according to any of embodiment 7 or 8, wherein the pharmaceutical composition of claim 5 or 6 is administered between 6 hours and 15 minutes, preferably between 3 hours and 25 minutes, before PET/CT or PET/MRI or SPECT/CT or SPECT/MRI imaging.
10. The method according to any of embodiments 7 to 9, wherein the pharmaceutical composition of claim 5 or 6 is administered at an amount between 150 MBq and 250 MBq.
11. The method of anyone of embodiments 7 to 10, wherein the pharmaceutical composition of embodiments 5 or 6 is suitable for injection, preferably a bolus injection.
12. The method of any one of embodiments 8 to 11, wherein a therapeutically efficient amount of a pharmaceutical composition of any one of embodiments 1 to 3 is administered at least two weeks after step (i) of administering the imaging agent.
13. A pharmaceutical composition of a radiolabelled αvβ3 and/or αvβ5 integrins antagonist which comprises:
   [ii] a radiolabelled αvβ3 and/or αvβ5 integrins antagonist of formula I:

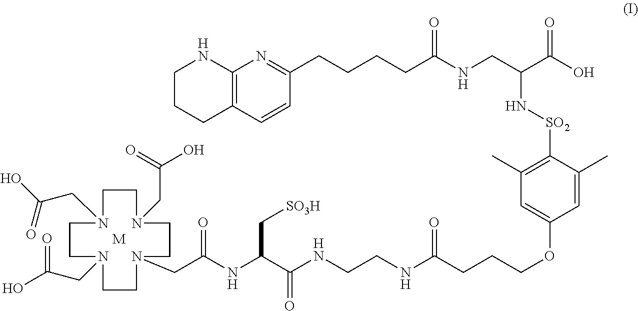

wherein:
M is a radiometal suitable for therapy, preferably 177-Lutetium, and
[ii] one or more pharmaceutically acceptable excipients.
14. The pharmaceutical composition of embodiment 13, wherein said radiometal is present at a concentration providing a volumetric radioactivity of at least 10 mCi/mL (370 MBq/mL), preferably at least 15 mCi/mL (555 MBq/mL), more preferably at least 20 mCi/mL (740 MBq/mL) in said pharmaceutical composition.
15. The pharmaceutical composition of embodiment 13 or 14, wherein molar ratio between the radiolabelled αvβ3 and/or αvβ5 integrins antagonist of formula I and the radiometal is at least 1.3, preferably between 1.3 and 1.8, more preferably about 1.5.
16. The pharmaceutical composition of embodiments 13-15, wherein the one or more pharmaceutically acceptable excipients is (are) selected from stabilizers against radiolytic degradation, solvents, sequestering agents, pH adjusters and mixtures thereof.
17. The pharmaceutical composition of embodiments 13-16, wherein the one or more pharmaceutically acceptable excipients are at least two stabilizers against radiolytic degradation selected from gentisic acid or salts thereof, ascorbic acid or salts thereof, methionine, histidine, melatonine, and Se-methionine, preferably selected from gentisic acid or salts thereof and ascorbic acid or salts thereof, more preferably said at least two stabilizers are gentisic acid and sodium ascorbate.
18. The pharmaceutical composition of embodiments 13-17, wherein a ratio between gentisic acid and sodium ascorbate is between 1:100 and 1:200, preferably between 1:110 and 1:150, more preferably between 1:120 and 1:135, and even more preferably about 1:128.
19. The pharmaceutical composition of embodiments 17 or 18, wherein said gentisic acid or salts thereof, preferably gentisic acid, is present in a concentration of least 0.30 mg/mL, preferably at least 0.35 mg/mL, and more preferably at least 0.38 mg/mL, typically between 0.30 mg/mL and 0.40 mg/mL, preferably between 0.38 mg/mL and 0.40 mg/mL, more preferably about 0.39 mg/mL.
20. The pharmaceutical composition of embodiments 17-19, wherein said ascorbic acid or salts thereof, preferably sodium ascorbate, is present in a concentration of at least 40 mg/mL, preferably at least 45 mg/mL, typically between 40 mg/mL and 75 mg/mL, preferably between 45 mg/mL and 65 mg/mL, more preferably about 50 mg/mL.
21. The pharmaceutical composition of embodiments 17-20, wherein said gentisic acid or salts thereof is present in a concentration between 0.30 mg/mL and 0.40 mg/mL, preferably between 0.38 mg/mL and 0.40 mg/mL, and ascorbic acid or salts thereof can be present at a concentration between 40 mg/mL and 75 mg/mL, preferably between 45 mg/mL and 65 mg/mL.
22. The pharmaceutical composition of embodiments 13-21, having a radiochemical purity higher than 95% up to 72 hours and 120 hours, preferably equal to or higher than 97% up to 72 hours and equal to or higher than 96.5% up to 120 hours.
23. The pharmaceutical composition of embodiments 13-22, wherein the one or more pharmaceutically acceptable excipients are at least two stabilizers against radiolytic degradation and at least one other pharmaceutically acceptable excipient selected from solvent, sequestering agent, pH adjuster and mixtures thereof, preferably mixtures thereof.
24. The pharmaceutical composition of embodiment 23, wherein said solvent is water for injection and/or saline solution, preferably water for injection.
25. The pharmaceutical composition of embodiment 23, wherein said sequestering agent is diethylentriaminepentaacetic acid (DTPA) or a salt thereof, preferably at a concentration between 50 and 200 μg/mL, more preferably between 75 and 150 μg/mL, and even more preferably about 100 μg/mL.
26. The pharmaceutical composition of embodiment 23, wherein said pH adjuster is acetic acid and/or sodium acetate, preferably acetic acid and sodium acetate.
27. The pharmaceutical composition of embodiment 26, wherein said acetic acid is present at a concentration between 0.20 mg/mL and 0.40 mg/mL, preferably about 0.30 mg/mL and said sodium acetate is present at a concentration between 0.30 mg/mL and 0.50 mg/mL, preferably about 0.41 mg/mL.
28. The pharmaceutical composition of embodiments 13-27, wherein the pharmaceutical composition is an aqueous solution, preferably a solution for infusion.
29. The pharmaceutical composition of embodiments 13-28, wherein the pharmaceutical composition is produced at commercial scale manufacturing, preferably at a batch size of at least 1 Ci (37 GBq), more preferably 2 Ci (74 GBq).
30. The pharmaceutical composition of embodiments 13-28, wherein the pharmaceutical composition is for commercial use.

### FIGURES

FIG. 1: ⁶⁸Ga-Compound biodistribution in bladder, right and left kidney of male WM266 tumor xenograft bearing mice.

FIG. 2: ⁶⁸Ga-Compound biodistribution in other organs of male WM266 tumor xenograft bearing mice.

FIG. 3: ⁶⁸Ga-FF58 biodistribution in bladder, right and left kidney of female WM266 tumor xenograft bearing mice.

FIG. 4: ⁶⁸Ga-FF58 biodistribution in other organs of female WM266 tumor xenograft bearing mice.

### EXAMPLES

### Example 1: In Vivo Distribution of the [68Ga]-Compound in Subcutaneous WM266 Melanoma Cancer Model

Nude, male and female athymic mice bearing the WM266 melanoma tumor were intravenously injected with ⁶⁸Ga-Compound (˜100-150/uCi 320-480 pmol for males, ˜100-150 uCi/127-191 pmol for females, corresponding to 3.7-5.55 MBq). Animals were sacrificed in groups of four at 30 min, 1 h, 2 h and 4 h post injection. Tumor and organs of interest were collected, weighed and measured for radioactivity with a gamma-counter. The intestines and stomach were not emptied of their contents. Data were calculated as a percentage of the injected dose per gram of tissue (% ID/g).

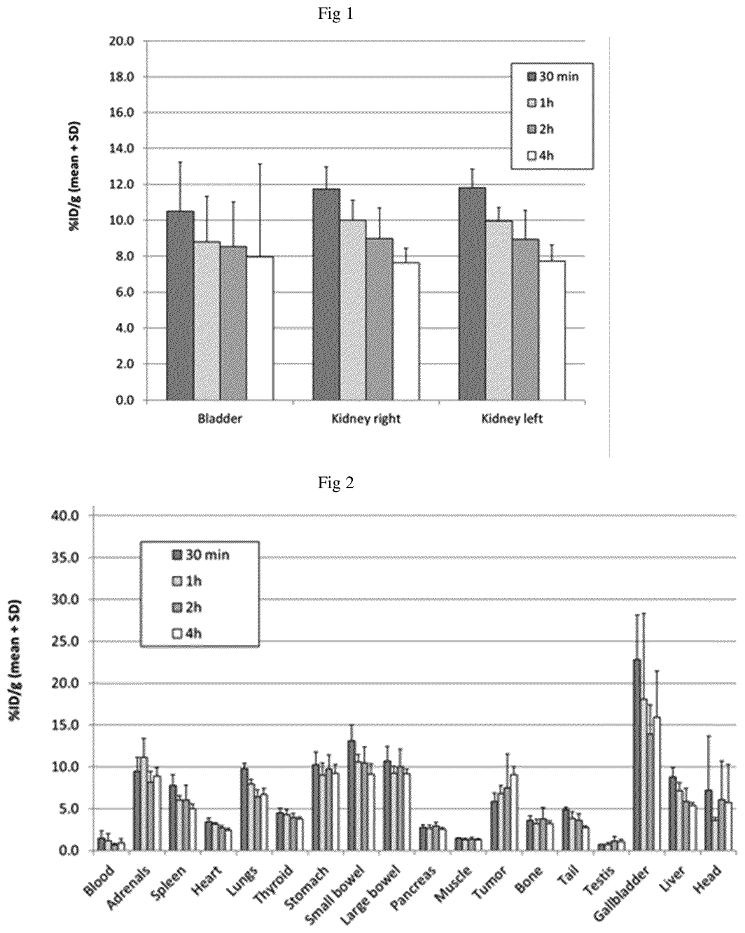

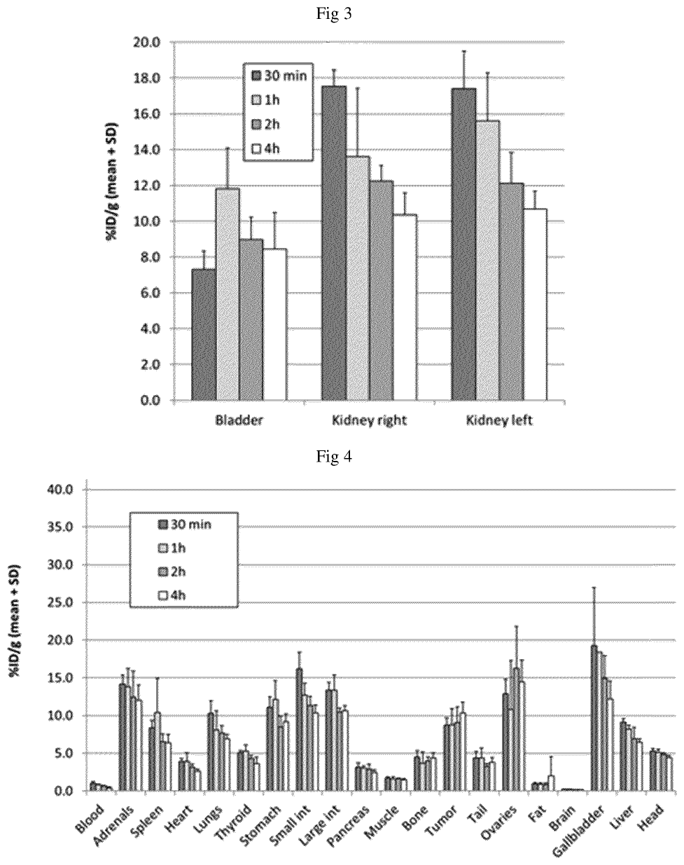

As can be seen in FIGS. 1, 2, 3 and 4, tumor uptake was around 6-9% and 8-10% in male and female mice, respectively, and did not decrease over time, remaining unchanged even at 4 hours post injection. Despite the detection of background uptake in normal organs at 30 min, it had cleared moderately by 4 h. Uptake in the kidneys and intestine confirmed renal and partial hepatic excretion in both male and female animals.

The preclinical data presented using 68Ga-Compound as a ligand, show a suitable profile of the molecule for its use as radio diagnostic agent in human patients.

The lack of pharmacological effects, the high affinity for αvβ3/αvβ5 and the absence of affinity for other receptors ensure that the molecule selectively binds to the target and carries the radioisotope for imaging detection without any physiological or pharmacological alteration of the tumor tissue that overexpresses the target. Moreover, in vivo studies in relevant tumor animal models demonstrate a favourable biodistribution of the radioligand in the tumor mass overexpressing the target, with suitable clearance in other organs and imaging performance.

### Example 2: Use of the Theranostic Pair [⁶⁸Ga]/[¹⁷⁷Lu]-Compound in Adult Patients with Melanoma Suspected to Overexpress αvβ3 and αvβ5 Integrins

[⁶⁸Ga]-Compound is administered at a fixed dose of 3 MBq/kg/injection±10%, with no less than 150 MBq and no more than 250 MBq/injection.

Static whole-body [⁶⁸Ga]-Compound PET imaging at 30 min (±15 min), 1 h (±15 min) and 2 h (±30 min) post-injection, and conventional imaging scan (high resolution CT or MRI) is acquired either as part of the [⁶⁸Ga]-Compound PET scan (joint acquisition as PET/CT with high resolution diagnostic CT or joint acquisition as PET/MRI with high resolution MRI) or separately within 24 h prior to or after [⁶⁸Ga]-Compound injection.

Then the preliminary targeting properties of [⁶⁸Ga]-Compound in patients are characterized by establishing the:
Number and location of tumor lesions detected by [⁶⁸Ga]-Compound overall and for each tumor type, and
Calculation of the ratio tumor/background standard uptake value (SUV) and % injected dose per gram of tissue (ID/g) and calculation of absorbed dose (mGy/MBq) in tumor overall and for each tumor type.

When 5%, 10%, 20%, 30%, 40%, 50%, 60%, 70% or 80% of the lesions identified by conventional imaging are also identified by [⁶⁸Ga]-Compound (100%×double positive/total number of lesions identified in conventional imaging) the patient is selected for the treatment with [¹⁷⁷Lu]-Compound.

[¹⁷⁷Lu]-Compound is administered at a fixed dose of 200 MBq/kg/injection±10% every 6-8 weeks up to a cumulative dose which is tolerated by the patient (maximum 800 GBq).

Results demonstrate that intravenous administration of [¹⁷⁷Lu]-Compound has a positive effect on melanoma patient.

### Example 3: Use of the [⁶⁸Ga]-Compound in a Phase I Single Dose Study in Adult Patients with GBM or Brain Metastasis (HER2+ Breast Cancer) Suspected to Overexpress αvβ3 and αvβ5 Integrins

This is a phase I, single dose study of [⁶⁸Ga]-Compound to characterize the imaging properties, safety, bio-distribution and dosimetry of [⁶⁸Ga]-Compound in adult patients with relapsed or refractory bevacizumab naive GBM who have failed prior radiation therapy or newly diagnosed brain metastasis from HER2+ breast cancer, suspected to overexpress αvβ3 and αvβ5 integrins.

Available preclinical data with the [⁶⁸Ga]-Compound supports the development in GBM where a remarkable 80% of GBM tumors are αvβ3 and αvβ5 integrin positive (Schittenhelm 2013).

The indication of brain metastasis is also selected for the study to enable the visualization of extra-cranial metastasis from the primary tumor. In order to explore a homogenous population, HER2+ breast cancer is selected as the primary origin of brain metastasis.

A total of 40 patients is enrolled into the study which is made up of parts A and B. Approximately 20 patients in GBM (male and female) and 20 patients in brain metastasis (HER2+ breast cancer) are included. Of these 40 patients, approximately 12 are included into the dosimetry sub-group and for which at least 1 dosimetry patient must be female.

Part A of the study enrolls 12 patients (6 relapsed/refractory GBM patients and 6 brain metastasis (HER2+) patients). Of these, approximately 3 patients per indication participates in the dosimetry sub-study. Of the 6 relapsed/refractory GBM bevacizumab naïve patients, 3 have surgical intent.

Enrolment for each indication runs in parallel and is evaluated independently from each other for continuation into part B. Once the patients from Part A enroll in each arm, an internal preliminary review based on safety, bio-distribution and 68Ga uptake is done. The continuation of patient enrollment of both arms into Part B of the study does not take place if there is no 68Ga-Compound positive patient in each indication.

Part B of the study enrolls 14 relapsed or refractory GBM patients and 14 brain metastasis (HER2+BC) patients. The dosimetry sub-group of part B includes approximately 3 patients per indication.

All patients enroll in the study receive a single dose of [⁶⁸Ga]-Compound. Based on pre-clinical data, the recommended dose for [68Ga]-Compound is 3 MBq/kg/injection±10%, with no less than 150 MBq and no more than 250 MBq/injection. This takes into consideration the half-life of the radionuclide (68 minutes for 68Ga), as well as the foreseen imaging and blood sampling schedule in this early phase study (up to 5 h post dose in the dosimetry sub-group), and which is considered adequate to obtain good quality imaging.

### Imaging Assessments

For all patients:
   Static whole-body [⁶⁸Ga]-Compound PET imaging at 30 min (±15 min), 1 h (±15 min) and 2 h (±30 min) post-injection, and
   Conventional imaging scan (high resolution CT or MRI) acquired either as part of the [⁶⁸Ga]-Compound PET scan (joint acquisition as PET/CT with high resolution diagnostic CT or joint acquisition as PET/MRI with high resolution MRI) or separately within 24 h prior to or after [⁶⁸Ga]-Compound injection.
For dosimetry sub-group only:
   Additional whole body dynamic [⁶⁸Ga]-Compound PET imaging between 0 to 15 min post-injection, and
   Additional static whole body [⁶⁸Ga]-Compound PET imaging acquisitions between 30 minutes to 4 h post-injection.

The PET imaging time points in Part A of the study is selected in order to evaluate the optimal time interval for imaging acquisition after [⁶⁸Ga]-Compound administration in Part B.

Conventional imaging, as a gold standard, allows the comparison of both imaging techniques ([⁶⁸Ga]-Compound PET and high resolution CT/MRI) and to identify the percentage of identical lesions identified by both methods for each patient as well as the number and location of discordant lesions identified with only [⁶⁸Ga]-Compound PET or with conventional imaging.

The overall sample size of approximately 40 patients (20 GBM and 20 brain metastases patients) ensures that sufficient αvβ3 and αvβ5 positive subjects are available to support initial assessment of imaging properties and design of subsequent clinical studies.

### Example 4: Identification of the Suitable Formulation for [¹⁷⁷Lu] Compound

The formulation development has been performed with the aim of identifying the reaction mixture composition able to allow a simple labelling of the Compound based on direct addition of ¹⁷⁷LuCl3 without any additional purification step.

The initial development tests were performed in order to identify a formulation capable of producing consistently a solution of [¹⁷⁷Lu]Lu-FF58 with adequate radiochemical purity and stability up to 120 hours.

The target characteristics of the ¹⁷⁷Lu-radiolabelled FF58 are as follow:
¹⁷⁷Lu-FF58 (HPLC)→≥97.0% (at t0h)
¹⁷⁷Lu-FF58 (HPLC)→≥95.0% (up to 120 h)
¹⁷⁷Lu free+¹⁷⁷Lu-DTPA (HPLC)→≤3.0%
¹⁷⁷Lu fragments (HPLC)→≤3.0%
¹⁷⁷Lu free+¹⁷⁷Lu colloidal (iTLC)→≤3.0%
¹⁷⁷Lu-DTPA (iTLC)→≤3.0%

The main components of the final formulation are as follow:
1. Active Pharmaceutical Ingredient
2. Antioxidant Agents (Gentisic Acid and Ascorbic Acid)
3. Sequestering Agent (DTPA)
4. Buffer Agent (Sodium Acetate and Acetic Acid)

1) FF58 Amount and FF58:Lu Molar Ratio Investigation

An amount of 2 milligrams of FF58 was selected for the preparation of a 200 mCi dose for potential batch size of 2 Ci.

The effect of the molar ratio between FF58 and ¹⁷⁷Luon the radiochemical purity was investigated in order to identify the suitable range of values which allows to meet all the target characteristics of the radiolabelled product. Increasing nanomolar ratios (FF58:Lutetium) were tested for the labeling in order to identify the minimum amount required to have a ¹⁷⁷Lu percentage complexation above 97% in HPLC (¹⁷⁷LuFree≤3%).

The specific activity (SA) corresponds to the ratio between the radioactivity and the sum of all the Lu isotopes present in solution. Since all these isotopes compete together for complexation with the DOTA-moiety, it is important to define the maximum amount of Lu, or the minimum SA value, which permit to guarantee the total coordination of the ¹⁷⁷Lu3+ ions.

The tests were performed using 10 mCi of ¹⁷⁷LuCl3 solution and adding the suitable amount of Reaction Buffer; once the radiolabelling step was concluded, the solution was formulated in the same conditions as [¹⁷⁷Lu]Lu-FF58.

Results, summarized in the table below, demonstrated that a ratio (API:Lu) of 1.30 was sufficient to guarantee the complexation of the DOTA moiety with the radioisotope resulting in a radiochemical purity within the acceptance criteria.

TABLE 1
FF58:Lu molar ratio

| Ratio | HPLC | |
|---|---|---|
| API/Lu | ¹⁷⁷Lu-fragments | ¹⁷⁷LuFF58 |
| 1.1 | 8.10% | 91.90% |
| 1.3 | 2.46% | 97.54% |
| 1.5 | 0.73% | 99.27% |
| 1.8 | 0.68% | 99.32% |

A molar ratio of 1.50 was selected for the development in order to guarantee sufficient robustness to the process. Once defined this value, it has been calculated the minimum specific activity of ¹⁷⁷LuCl₃ which allows to consistently guarantee incorporation of 177Lu3+ above 97% considering 2 mg as target amount of chemical precursor for the 2 Ci batch size. The minimum specific activity calculated is 10.00 Ci/mg, taking into account a safety margin 11.00 Ci/mg was selected.

2) Antioxidant Agent: Sodium Ascorbate and Gentisic Acid Amount

Preliminary tests performed on FF58 demonstrated that the molecule shows a good resistance to the radiation stress. Additional tests were performed in order to evaluate the radiolysis also in more stressful conditions such as 40° C. storage temperature and rolling, decreasing antioxidant agent (sodium ascorbate and gentisic acid) amounts. The radiochemical purity (RCP) was monitored at release time and at expiration time by Radio-RP-HPLC.

The results collected are reported in the table below.

TABLE 2
Antioxidant agents

| | | | | | | | [¹⁷⁷Lu]Lu- | |
|---|---|---|---|---|---|---|---|---|
| | | Volumetric | Gentisic | Sodium | | [¹⁷⁷Lu]Lu- | FF58 | [¹⁷⁷Lu]Lu- |
| Batch | Volume | Activity | Acid | Ascorbate | Storage | FF58 | At T5 d | FF58 |
| number | mL | (mCi/mL) | mg/mL | mg/mL | condition | t0 h (HPLC) | (HPLC) | T6 d (HPLC) |
| LI210402A | 3.5 | 21 | 0.384 | 41.022 | Room | 99.8 | 97.6 | 96.6 |
| | | | | | Temperature | | | |
| LI210402A | 3.5 | 21 | 0.384 | 41.022 | 40° C. | 99.8 | 95.4 | — |
| LI210402A | 3.5 | 21 | 0.384 | 41.022 | Rolling | 99.8 | 96.3 | — |
| LI210402A | 3.5 | 21 | 0.377 | 63.557 | Room | 99.8 | 97.5 | — |
| | | | | | Temperature | | | |
| LI210402A | 15.0 | 21 | 0.377 | 63.557 | 40° C. | 99.8 | 97.9 | 97.4 |
| LI210402A | 3.5 | 21 | 0.375 | 73.583 | Room | 99.8 | 97.8 | 97.9 |
| | | | | | Temperature | | | |
| LI210402A | 3.5 | 21 | 0.375 | 73.583 | 40° C. | 99.8 | — | 96.9 |
| LI210402A | 3.5 | 21 | 0.375 | 73.583 | Rolling | 99.8 | — | 97.9 |
| LI210514A | 3.5 | 21 | 0.132 | 48.920 | Room | 99.9 | 97.3 | 95.6 |
| | | | | | Temperature | | | |
| LI210514A | 3.5 | 21 | 0.132 | 48.920 | 40° C. | 99.9 | 95.8 | 94.4* |
| LI210514A | 3.5 | 21 | 0.132 | 48.920 | Rolling | 99.9 | 95.6 | 95.0 |
| LI210514A | 3.5 | 21 | 0.210 | 48.233 | Room | 99.9 | 97.2 | 96.2 |
| | | | | | Temperature | | | |
| LI210514A | 3.5 | 21 | 0.210 | 48.233 | 40° C. | 99.9 | 96.4 | 94.5* |
| LI210514A | 3.5 | 21 | 0.210 | 48.233 | Rolling | 99.9 | 96.5 | 95.5 |
| LI210514A | 3.5 | 21 | 0.210 | 38.334 | Room | 99.9 | 97.4 | 96.7 |
| | | | | | Temperature | | | |
| LI210514A | 3.5 | 21 | 0.210 | 38.334 | 40° C. | 99.9 | 94.7* | — |
| LI210514A | 3.5 | 21 | 0.210 | 38.334 | Rolling | 99.9 | 95.9 | 94.6* |

*the results is out of specification

Based on the above results, it is demonstrated that gentisic acid and sodium ascorbate in a concentration lower than 300 μg/mL of gentisic acid and 40 mg/mL of sodium ascorbate are not adequate to guarantee a radiochemical purity of the Finished Product compliant with the quality specification of 95.0% at the end of shelf life.

The holding time of the drug substance solution into the intermediate vial after synthesis is a critical parameter for the radiochemical purity (RCP) of the Drug Product, therefore additional test were performed increasing the holding up to 1 h.

The results collected are reported in the table below.

TABLE 3
Antioxidant agents - 1 hour holding time

| | | | | | | | | [¹⁷⁷Lu]Lu- | |
|---|---|---|---|---|---|---|---|---|---|
| | | Volumetric | Gentisic | Sodium | | | [¹⁷⁷Lu]Lu- | FF58 | [¹⁷⁷Lu]Lu- |
| Batch | Volume | Activity | Acid | Ascorbate | Storage | | FF58 | At T5 d | FF58 |
| number | mL | (mCi/mL) | mg/mL | mg/mL | condition | | t0 h (HPLC) | (HPLC) | T6 d (HPLC) |
| LI210618A | 3.5 | 21 | 0.384 | 44.06 | Room | | 99.8 | 96.9 | 95.9 |
| | | | | | Temperature | | | | |
| LI210618A | 25.0 | 21 | 0.384 | 44.06 | Room | | 99.8 | 98.4 | — |
| | | | | | Temperature | | | | |
| LI210618A | 3.5 | 21 | 0.384 | 44.06 | 40° | C. | 99.8 | 96.8 | 94.5* |
| LI210618A | 25.0 | 21 | 0.384 | 44.06 | 40° | C. | 99.8 | 98.1 | — |
| LI210618A | 3.5 | 21 | 0.384 | 44.06 | 2-8° | C. | 99.8 | 98.5 | — |

| | | | | | | | | |
|---|---|---|---|---|---|---|---|---|
| LI210618A | 3.5 | 21 | 0.384 | 44.06 | Rolling | 99.8 | 98.1 | 96.4 |
| LI210618A | 3.5 | 21 | 0.384 | 44.06 | Up Side Down | 99.8 | 95.7 | 94.9* |

*the results is out of specification

Despite the increase of the holding time, the results reported above show the compliance of the quality specification of finished product up to the end of shelf life.

The minimum amount of antioxidants into the Drug Product was set up as follow:
Gentisic Acid=0.30 mg/mL;
Sodium Ascorbate=40.00 mg/mL.

And the target amount was set up as follow:
Gentisic Acid=0.39 mg/mL;
Sodium Ascorbate=50.00 mg/mL.

### Example 5: Final Formulation and Detailed Composition

Based on the results described in Example 4, the [¹⁷⁷Lu]Lu-FF58 formulation selected for the validation activities of 2 Ci batch size process is the following:

TABLE 4
Final Formulation

| | | |
|---|---|---|
| Component | Function | Final Amount |

Active Ingredients

| | | | |
|---|---|---|---|
| ¹⁷⁷Lu-FF58 | 777 | MBq/mL | Radiopharmaceutical |
| | | | active ingredient |
| FF58 | 21.0 | μg/mL | Peptide Content |

Other Ingredients

| | | |
|---|---|---|
| Acetic Acid | 0.30 | pH adjuster |
| Sodium Acetate | 0.41 | pH adjuster |
| Sodium Ascorbate | 50.00 | Antioxidant |
| Gentisic Acid | 0.39 | Antioxidant |
| Pentetic Acid | 0.10 | Chelating agent |
| (DTPA) | | |
| WFI | — | Solvent |

The formulation has been tested and has demonstrated to consistently produce the [¹⁷⁷Lu]Lu-FF58 with adequate radiochemical purity and stability.

The results are reported below:

Table summarizes the radiochemical purity results obtained for the [¹⁷⁷Lu]Lu-FF58 2 Ci batch.

TABLE 7
Quality control results RCP (LI210618A)

| | | Storage | | RCP | RCP | RCP | RCP |
|---|---|---|---|---|---|---|---|
| OC | Volume | temp | | HPLC at | iTLC at | HPLC at | HPLC at |
| vial | (mL) | (° C.) | | t0 (%) | t0 (%) | t72 h (%) | t120 h (%) |
| 9 | 3.5 | 2-8° | C. | 99.8 | 100.0 | 98.9 | 98.5 |
| 1 | 3.5 | RT | | | | 98.3 | 96.9 |
| 3 | 3.5 | 40° | C. | | | 97.9 | 96.8 |

1. A method for the treatment of tumors overexpressing αvβ3 and/or αvβ5 integrins selected from glioblastoma, head and neck cancer, gastroesophageal adenocarcinoma, colorectal cancer, breast cancer, small cell lung cancer, non-small cell lung cancer, malignant melanoma, gastric cancer, and pancreatic cancer, comprising administering an effective amount of a pharmaceutical composition to a human subject in need thereof, wherein said pharmaceutical composition comprises
[i] a radiolabelled αvβ3 and/or αvβ5 integrins antagonist of formula I: 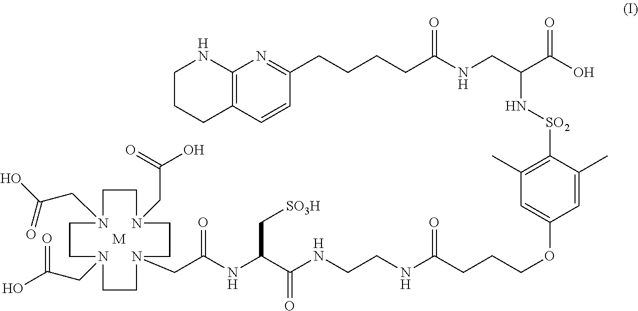
wherein:
M is a radiometal suitable for therapy, and
[ii] one or more pharmaceutically acceptable excipients,
wherein said subject has been selected for the treatment by SPECT/CT or PET/CT or SPECT/MRI or PET/MRI imaging with the same αvβ3 and/or αvβ5 integrins antagonist as defined for the treatment but wherein M is a radiometal suitable for imaging.

2. The method according to claim 1, wherein tumors overexpressing αvβ3 and/or αvβ5 integrins are selected from glioblastoma, malignant melanoma, brain metastasis from breast cancer and melanoma.

3. The method according to claim 1, wherein the pharmaceutical composition is suitable for injection.

4. The method according to claim 1, wherein, a subject has been selected for the treatment by evaluating [⁶⁸Ga]-labeled αvβ3 and/or αvβ5 integrins antagonist uptake in the lesions as determined by PET/MRI or PET/CT or SPECT/CT or SPECT/MRI imaging in said subject.

5. A method for determining whether a subject can be selected for a treatment with radiolabelled αvβ3 and/or αvβ5 integrins antagonist for treating tumors overexpressing αvβ3 and/or αvβ5 integrins, said tumors are selected from glioblastoma, head and neck cancer, colorectal cancer, breast cancer, small cell lung cancer, non-small cell lung cancer, malignant melanoma, gastric cancer, pancreatic cancer, prostate cancer and brain metastasis, said method comprising administering an effective amount of a pharmaceutical composition of a radiolabelled αvβ3 and/or αvβ5 integrins antagonist, as a imaging agent for PET/CT or PET/MRI or SPECT/CT or SPECT/MRI imaging, to a human subject in need thereof, wherein said pharmaceutical composition comprises
[i] a radiolabelled αvβ3 and/or αvβ5 integrins antagonist of formula I: 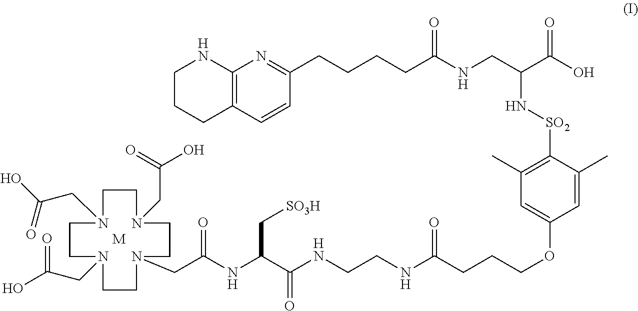
wherein:
M is a radiometal for use as imaging agent in SPECT/CT or PET/CT or SPECT/MRI, PET/MRI imaging, and
[ii] one or more pharmaceutically acceptable excipients,
wherein said subject is selected for the treatment by evaluating uptake of said radiolabelled αvβ3 and/or αvβ5 integrins antagonist in said tumors overexpressing αvβ3 and/or αvβ5 integrins by PET/CT or PET/MRI or SPECT/CT or SPECT/MRI imaging in said subject.

6. The method according to claim 5, wherein said tumors overexpressing αvβ3 and/or αvβ5 integrins are selected from glioblastoma, gastroesophageal adenocarcinoma, malignant melanoma, pancreatic ductal adenocarcinoma, brain metastasis from breast cancer and melanoma.

7. The method according to claim 5, said method comprising the steps of:
(i) administering an effective amount of the pharmaceutical composition as as an imaging agent for imaging the uptake of the radiolabelled αvβ3 and/or αvβ5 integrins antagonist in said tumor lesions,
(ii) acquiring an image by PET/MRI or PET/CT or SPECT/CT or SPECT/MRI of said patient, and
(iii) determining the uptake of the radiolabelled αvβ3 and/or αvβ5 integrins antagonist based on the image acquired at step (ii) in said tumor lesions.

8. The method of claim 7, further comprising a step of treating tumors overexpressing αvβ3 and/or αvβ5 integrins by administering a therapeutically efficient amount of a pharmaceutical composition which comprises
[i] a radiolabelled αvβ3 and/or αvβ5 integrins antagonist of formula I: 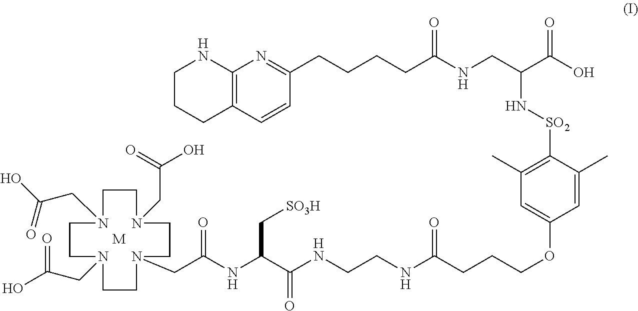
wherein:
M is a radiometal suitable for therapy, and
[ii] one or more pharmaceutically acceptable excipients.

9. The method according to claim 7, wherein the pharmaceutical composition is administered between 6 hours and 15 minutes, preferably between 3 hours and 25 before PET/CT or PET/MRI or SPECT/CT or SPECT/MRI imaging.

10. The method according to claim 7, wherein the pharmaceutical composition is administered in an amount between 150 MBq and 250 MBq.

11. The method of claim 7, wherein the pharmaceutical composition is suitable for injection.

12. The method of claim 7, wherein a therapeutically effective amount of a pharmaceutical composition is administered at least two weeks after step (i) of administering the imaging agent wherein the pharmaceutical composition comprises
[i] a radiolabelled αvβ3 and/or αvβ5 integrins antagonist of formula I: 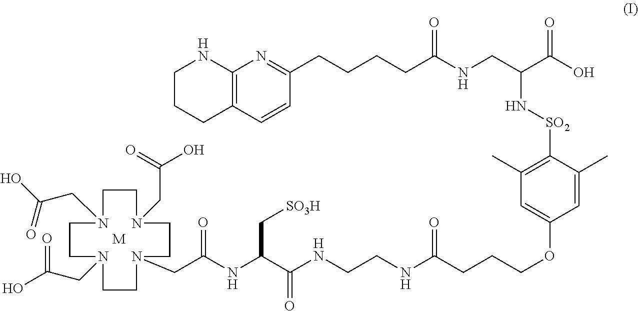
wherein:
M is a radiometal suitable for therapy, and
[ii] one or more pharmaceutically acceptable excipients.

13. The method according to claim 1, wherein the radiometal M suitable for therapy is 177-Lutetium.

14. The method according to claim 1, wherein the radiometal M suitable for imaging is 68-Gallium, 67-Gallium or 64-Copper.

15. The method according to claim 1, wherein the radiometal M suitable for imaging is 68-Gallium.

16. The method according to claim 1, wherein tumors overexpressing αvβ3 and/or αvβ5 integrins is a malignant melanoma.

17. The method according to claim 1, wherein the pharmaceutical composition is suitable for infusion.

18. The method according to claim 5, wherein the radiometal M is 68-Gallium.

19. The method according to claim 5, wherein tumors overexpressing αvβ3 and/or αvβ5 integrins is a malignant melanoma.

20. The method according to claim 7, wherein the pharmaceutical composition is administered between 3 hours and 25 minutes, before PET/CT or PET/MRI or SPECT/CT or SPECT/MRI imaging.
